(19) United States
(12) Patent Application Publication
(10) Pub. No.: US 2025/0090031 A1
(43) Pub. Date: Mar. 20, 2025

(54) Smart Tourniquet

(71) Applicant: Fiomet Ventures, Inc., Cincinnati, OH (US)

(72) Inventor: Scott Rapp, Cincinnati, OH (US)

(21) Appl. No.: 18/964,361

(22) Filed: Nov. 30, 2024

Related U.S. Application Data
(63) Continuation-in-part of application No. 17/163,353, filed on Jan. 30, 2021, now abandoned.
(63) Continuation-in-part of application No. 18/762,907, filed on Jul. 3, 2024.
Application No. 18/762,907 is a continuation of application No. 17/169,411, filed on Feb. 6, 2021, now Pat. No. 12,036,002.
Application No. 17/169,411 is a continuation of application No. 15/289,071, filed on Oct. 7, 2016, now Pat. No. 10,973,413.
(60) Provisional application No. 62/969,067, filed on Feb. 1, 2020.
(60) Provisional application No. 62/238,522, filed on Oct. 7, 2015.

(51) Int. Cl. A61B 5/01; A41D 1/00; A61B 5/00; A61F 5/00; A61F 5/01; A61F 13/06

(52) U.S. Cl. CPC A61B 5/01; A41D 1/002; A61B 5/0053; A61B 5/6804; A61B 5/6824; A61B 5/6828; A61F 5/00; A61F 5/0104; A61F 13/06

(57) ABSTRACT

A multilayer smart tourniquet applied about a wound environment or injured tissue. The smart tourniquet can be supplied with sensors and micro heating and cooling units. The smart tourniquet can be utilized with or with a bladder proximate to the wound environment. Select embodiments of the tourniquet are adapted to act as synthetic muscle.

### PRIORITY

This Application is a Continuation-in-Part of application Ser. No. 17/163,353—Smart Tourniquet—, filed Jan. 30, 2021 that claims the benefit of Provisional Application 62/969,067—Wound Treatment Devices—filed Feb. 1, 2020 and also claims priority to Continuation application Ser. No. 18/762,907—Advanced Compression Garments and Systems—, filed Jul. 3, 2024; that claims priority to Continuation application Ser. No. 17/169,411—Advanced Compression Garments And Systems—, filed Feb. 6, 2021 (now U.S. Pat. No. 12,036,002, granted Jul. 16, 2024); that claims priority to U.S. application Ser. No. 15/289,071—Advanced Compression Garments And Systems—, filed Oct. 7, 2016 (now U.S. Pat. No. 10,973,413, granted Apr. 13, 2021) that claims the benefit of US Provisional Application No. 62/238,522, filed Oct. 7, 2015.

### BACKGROUND OF THE INVENTION

### A. Field of the Invention

The current smart tourniquet utilizes smart technology and compression structures that can assist in controlling venous and arterial bleeding. The tourniquet can also supply pressure to a soft tissue defect or wound. Use of smart technology provides real-time data sensed by pressure and other biometrics to the user. It is believed that use of the present smart tourniquet can reduce morbidity associated with post injury soft tissue and lessen mortality rates from penetrating injuries.

Time is the most critical factor from the onset of injury during battle to medical intervention. It has been estimated that over 90% of deaths in soldiers occur before they are evaluated at a stand-alone medical facility. Further, it is estimated that over 25% of these deaths may be preventable¹. Historically, extremity hemorrhage is responsible for over 8% of all battlefield deaths spanning from the Civil War until the 1990s. This led to multiple wings of the military branches to explore utilization of portable devices that can address uncontrolled hemorrhage from penetrating or blast injuries.

To combat preventable morbidity and mortality in extremity injuries, the use of tourniquets as a hemostatic intervention has taken various similar forms over the last few centuries. First implemented by Morel in 1674², the technology has largely gone unchanged since with little overall improvement to design. In 1873, Esmarch described use of a rubber bandage to exsanguinate the limb and application of a tourniquet made from rubber tubing³. It was reported that even as recently as 2001, tourniquet use was still limited to available resources such as bandanas, sticks, belts and other nearby materials¹. The basic premise for the tourniquet is to apply pressure over an axial or named artery, where twisting of a band about the soft tissue creates local compression and ischemia.

Medical professionals have been critical of tourniquet use due to multiple problems with its technology leading to the lack of universal adoption and acceptance in trauma and critical care even today. Focal application over a penetrating injury may not address the physical location of the hemorrhage. Arterial injuries often result in retraction of the vessel with migration quite proximally to the site of the injury. Tourniquet application in these scenarios may lead to venous hypertension as the veins are compressed. At the same time, unaffected collateral arteries may continue to remain patent despite external compression due to arterial lumen pressure exceeding the topical pressure applied. Blood flow may continue to travel past the tourniquet into the distal extremity. However, since the venous system is more easily compressed, returning blood flow may not be able to travel back through the tourniquet and thus resulting in extremity venous hypertension as blood volume increases distal to the tourniquet. Clinically this leads to increased lumen pressure in the venous system which gradually builds up over time. Once the lumen pressure in the veins exceeds the tourniquet compression forces, further hemorrhage or exsanguination can occur. Venous bleeding can appear or mimic arterial bleeding in nature and blood loss can be accelerated. Precise application of pressure over a damaged artery can be difficult on the battle field.

Prolonged ischemia times greater than 2 hours leads to observable and irreversible apoptosis in human cells under microscopy. Excessive pressure over a nerve can cause irreversible damage and chronic pain syndromes. Greater than 6 hours of tourniquet application usually leads to unsalvageable soft tissue loss resulting in amputation. Improper tourniquet application to a leg can be managed with a less morbid BKA (Below the Knee Amputation) or could require an AKA (Above the Knee Amputation). Median survival rates at 5 years have as low as 22.5% with an AKA and 37.8% with a BKA⁴. An AKA requires utilization of more resources and time for rehabilitation. It appears that post injury tissue preservation can improve multiple quality of life factors.

Further, to complicate tourniquet application, it was thought that letting the tourniquet down periodically would reduce these above adverse effects. However, re-perfusion injury from harmful ischemic metabolites and resultant venous hypertension can lead to further tissue loss. Venous thrombosis can limit blood return and increase hypertension.

In early 2000s, the Joint Combat Casualty Research (JCCR) provided independent research on the value of tourniquet utilization and increased survival. By 2005-2006, all Special Operations units were deployed with tourniquets. Newly designed tourniquets became commercially available and in 2008, the World War II tourniquet design was finally abandoned. The first widespread branded tourniquet was the CAT tourniquet or Combat Application Tourniquet. The CAT device is composed of durable nylon band around 3 cm across with a rigid rod fit through a loop to twist for tightening. Hook and loop fastener was added to prevent loosening. A second version called SAM Junctional Tourniquet (SJT) was implemented for junctional injuries to limbs. Results were favorable in the field. The 75th Ranger Regiment saw mortality rates drop to 0% for hemorrhage and saw preventable deaths account for just 3 percent of its fatalities, compared to 24 percent of all U.S. military fatalities⁵.

### B. Description of the Previous Art

Any discussion of references cited herein merely summarizes the disclosures of the cited references. Applicant makes no admission that any cited reference or portion thereof is relevant prior art. Applicant reserves the right to challenge the accuracy, relevancy and veracity of the cited references.

Publications or presentations that may indicate a state-of-art include:
1. US Army Institute of Surgical Research. Stopping the Bleed. How army surgeons brought tourniquets back into the medical mainstream. Excerpted from document presented Feb. 2, 2018.
2. Welling D R, McKay P L, Rasmussen T E, Rich N M. A brief history of the tourniquet. J Vasc Surg. 2012 January; 55(1):286-90.
3. Esmarch F. Ueber Kunstliche Bluterlee bei Operationen. Samml Klin Vortr 1873; 58:373-384.
4. Aulivola B, Hile C N, Hamdan A D, Sheahan M G, Veraldi J R, Skillman J J, Campbell D R, Scovell S D, LoGerfo F W, Pomposelli F B Jr. Major lower extremity amputation: outcome of a modern series. Arch Surg. 2004 April;139(4):395-9; discussion 399.
5. Kotwal R S, Montgomery H R, Kotwal B M, Champion H R, Butler F K Jr, Mabry R L, Cain J S, Blackbourne L H, Mechler K K, Holcomb J B. Eliminating preventable death on the battlefield. Arch Surg. 2011 December; 146(12):1350-8.

Patents and Published Patent Application that may indicate a state-of-art include: 1) U.S. Pat. No. 7,892,253—Esposito, et al. discloses a tourniquet and method of use (the CAT tourniquet); 2) U.S. Pat. No. 9,492,177—Saunders, et al. discloses a junctional tourniquet places focal pressure on the core body when vessels are injured proximal to the limb; 3) U.S. Pat. No. 4,469,099—McEwen discloses a pneumatic tourniquet; 4) U.S. Pat. No. 4,479,494—McEwen discloses an adaptive pneumatic tourniquet; 5) U.S. Pat. No. 5,439,477—McEwen discloses a tourniquet apparatus for applying minimum effective pressure; 6) U.S. Pat. No. 5,556,415—McEwen discloses a physiologic tourniquet for intravenous regional anesthesia; 7) U.S. Pat. No. 5,855,589—McEwen discloses a physiologic tourniquet for intravenous regional anesthesia; 8) U.S. Pat. No. 10,299,520—Shaffer et al discloses fabric-based items with environmental control elements; 9) U.S. Pat. No. 9,733,136—Seitz discloses a textile pressure sensor; 10) U.S. Pat. No. 4,860,748—Chiurco et al discloses thermal pattern generator for pain relief; 11) US Published Patent Application 20150073319—Holschuh et al discloses controllable compression textiles using shape memory alloys and associated products; 12) US Published Patent Application 20080262535—Gavriely et al discloses method and an apparatus for adjusting blood circulation in a limb; 13) US Published Patent Application 20200206035—Kantor et al discloses a medical bandage for the bead, a limb or a stump; 14) US Published Patent Application 20150173667—Ben Shalom discloses system and method of pressure mapping and 3-D subject repositioning for preventing pressure wounds; 15) US Published Patent Application 20150073326—hemostasis wound healing device for dynamic elastic injury site; 16) US Published Patent Application 20190262188—Reid Jr. discloses tension adjusting and stabilization system and method; 17) US Published Patent Application 20160008206—Devanaboyina discloses systems and methods for exerting force on bodies; 18) US Published Patent Application 20040108589—Heilbronner discloses and methods for exerting force on bodies; 19) US Published Patent Application 20110230747—Rogers et al discloses implantable biomedical devices on bioresorbable substrates; 20) US Published Patent Application 20200113773—Ramanan et al discloses compression apparatus and systems for circulatory disorders; 21) US Published Patent Application 20200246212—Lee discloses wearable electronic device, display device and control method therefor; 22) US Published Patent Application 20090287191—Ferren et al discloses circulator monitoring systems and methods and 23) US Published Patent Application 20060287621—Atkinson et al discloses medical compression devices and methods.

### SUMMARY OF THE INVENTION

Among other things, the current smart tourniquet can supply adjustable or constant pressures to a human wound. A communications module is detachable from the tourniquet. The communications module includes a transmitter or transceiver adapted to communicate with a Cloud server or computer, second computer such as a mobile computing device or a personal computer or any type of computing device remote from the communications module. Remote devices can be utilized to monitor or control pressures applied by the current smart tourniquet to cranial, venous, arterial or soft tissue wounds.

The current device and system embodiments can include a custom fitted tourniquet that can provide real time biometric feedback from multiple different sensors. Therapeutic interventions such as hemostasis, compression, adaptive heating and cooling and improved wound care optimization are possible with the current smart tourniquet.

To address hemorrhage following injury, among other things, the tourniquet has a custom foaming capability to adapt to any contour irregularity. The foaming agents are able to expand into the defect or around the defect to provide focal compression and improved hemostasis. By providing diffuse pressure over a greater surface area in and around the wound, less pressure can be used to obtain hemostatic control. With penetrating injuries, the foam will expand into this area providing a more appropriate mold of the defect than conventional tourniquets. A bladder interposition made of polyurethane or other polymeric materials may be implemented to ensure a watertight pressure application and enhanced rigidity to the foamed dressing. The bladder can be attached to the outward side of the inner layer of the tourniquet. When indicated by medical parameters, the current tourniquet provides control of external compression (more or less) supplied to the wound environment.

Within the scope of the current invention, biometric sensors can be positioned in inner layer, the bladder or both. In select embodiments of the tourniquet, biometric sensors such as pressure sensors can be placed at multiple locations of the invention. Use of one or more pressure sensors can provide alerts indications and/or alerts of acceptable, excessive or insufficient pressures. Insufficient pressures may result in excessive hemorrhage, low perfusion pressures, ischemia, secondary hypoxia leading to soft tissue, muscle, organ and neuronal death. Excessive pressures may result in downstream ischemia, venous hypertension, and focal soft tissue, axonal and cellular compromise. A biometric sensor communicates with communications module adapted to communicate with a computer distinct from the communications module. Communications module includes a transmitter or a transceiver allowing wireless communications with a remote computer that provides real real-time calculation/determination of the biometric(s) sensed. Communication modules can also include a memory, a microprocessor or processor and software (computing component) for calculating the biometric measurements sensed by the biometric sensors and controlling the tourniquet's heat/cooling semiconductors. Depending on medical treatment parameters, the remote computer or the communications module's computer module can be used to calculate sensed biometrics and control the heat/cooling semiconductors. In accordance with the present invention, the computer or computer module can calculate biometrics, including but not limited to, pressure, temperature, blood pressure, lactate levels, pH, sodium levels, potassium level, glucose levels, apoptotic factors, nitric oxide (NO) and SVO2 levels sensed by their respective sensors.

In selected embodiments of the current invention, semiconductors, such as micro-Peltier coolers, can be included in the inner layer, the bladder, the foam or any combination to provide heating or cooling. The direction of the current flow through micro-semiconductors allows for either cooling or warming. Power for the semiconductors and other components of the current tourniquet can be provided by batteries, a connection with an alternating current power source or a radio frequency energy supply.

Within the ambit of the current invention, micro-semiconductors can be rigid or flexible or a combination thereof. The semiconductors can utilize metallic thermal interfaces to dissipate heat or cold. Select preferred embodiments of the micro-semiconductors can utilize ferromagnetic foils that transition between paramagnetic and ferromagnetic states and can drive a metallic shuttle generating a squeezed-film cooling effect during oscillation. The thermal interfaces can induce vibration of fluids in proximity with the thermal interfaces. Select embodiments of the thermal interfaces can be provided with carbon nanotubes that can decrease thermal interface resistance. The carbon nanotubes can be grown on gadolinium foil. Use of carbon nanotubes can improve performance of the semiconductors.

It is believed that use of micro-semiconductors allows the application of different voltages and current across two distinct semiconductors, thereby generating a “heat flux” of hot or cold across the semiconductors' interfaces. Direction of current crossing the interfaces changes the generated heat flux from hot to cold or cold to hot such that the change the direction of current can result in a 25 degree Celsius deferential of temperature proximate the interfaces.

The current smart tourniquet can be sized to fit about body parts such as head, limbs or torso. Tourniquets adapted to be fitted about a body part can include a zipper spanning the length of tourniquets to provide easier application to the wound environment.

Within the scope of the current invention, tourniquets with prefabricated lumens can be pulled over the wound environment. For select preferred embodiments of tourniquets with prefabricated lumens, straps, hook and loop fasteners, hooks and catches, zippers or any combination thereof can be utilized to further secure the tourniquet about the wound environment and improve the tourniquet's post application stability.

The current custom tourniquet also has applications in veterinary medicine. By way of illustration, the smart tourniquet can provide focal compression to the lower extremity of a horse.

An aspect of the present invention is that the current tourniquet can be utilized on varying surface morphologies of humans, bovine, canine, equine, feline or zoo animals, etc.

Another aspect of the present invention is to provide a multilayer tourniquet including biometric sensors.

Yet another aspect of the present invention is to provide a tourniquet utilizing micro heating and cooling units.

Still another aspect of the present invention is to provide a tourniquet including synthetic muscle.

Yet still another aspect of the present invention is to provide a detachable communications module including a touchscreen displaying interactive data that allows the user to control one or more layers of the multilayer tourniquet.

Another aspect of the present invention is to provide a tourniquet adapted for use with expanding foam to conform either the foam or the tourniquet to the wound environment.

Still another aspect of the present invention is to provide a receptacle distinct from the layers of the tourniquet for releasably holding the communications module.

Yet still another aspect of the present invention is to provide a tourniquet including therapeutic zones.

A preferred embodiment of the current invention can be described as a tourniquet adapted for use about a wound environment or an injured tissue; the tourniquet comprising: a) a bladder, comprising: i) an inward side fitted to contact a contour of a portion the wound environment and adapted to receive an expansion fluid for supplying a preselected pressure to the wound environment, wherein: ii) the expansion fluid includes polymerized beads; iii) a first current flowing through graphene or carbon nanotubes causes the bladder to function as a first synthetic muscle for the wound environment; and iv) a port; b) an inner layer comprising an infrared radiation fabric or a bio ceramic-integrated fabric; the inner layer adapted to be proximate the bladder and the wound environment; the inner layer further comprising a hydrophobic adhesive polymer for impeding movement of the tourniquet about the wound environment; c) an array comprising: i) a group of first sensors conformable to the contour of a portion the wound environment and a section of the bladder, wherein the group of first sensors is adapted to sense pressures associated with the wound environment; and ii) a communications junction comprising a first magnetic source; d) a plurality of semiconductors comprising thermal interfaces and ferromagnetic properties connected to a wound environment facing side of the bladder, wherein dependent on a direction of a second current flowing through the thermal interfaces, the second current causes at least some of the plurality of semiconductors to heat or cool an area of the wound environment associated with the at least some of the plurality of semiconductors' footprints; e) an outer layer surrounding an outward side of the inner layer; f) a detachable communications module adapted for connection to the communications junction comprising a housing comprising: i) a first interface magnetically reciprocating with the communications junction; ii) a touchscreen visible to a user; and iii) a combination of components comprising a microprocessor, a memory, a visual graphics unit, an audio unit, a transmitter or transceiver and a software controlling the detachable communications module; g) the software: i) communicating with the group of first sensors and the plurality of semiconductors; and ii) controlling the microprocessor, the memory, the visual graphics unit, the audio unit, the transmitter or the transceiver and visual displays projected by the touchscreen, wherein the software controlling the detachable communications module is located on the detachable communications module, a Cloud server or a second computer remote from the detachable communications module, or a combination thereof; h) the transmitter or transceiver adapted for wireless communications, via an available wireless cellular network, an IEEE 802.11 protocol or military protocol with a Cloud server or a second computer remote from the communications module; and i) circuitry providing connections between the detachable communications module, the communications junction, the group of first sensors and the plurality of semiconductors and a power source for the detachable communications module.

Another preferred embodiment of the current invention can be described as a tourniquet adapted for use about a wound environment; the tourniquet comprising: a) an inner layer comprising: i) an infrared radiation fabric or a bio ceramic-integrated fabric; ii) a plurality of sensors attached to the inner layer, wherein at least some of the plurality of sensors sense pressures associated with the wound environment; and ii) a port adapted to receive and distribute expansion fluids, including polymerized beads, creating a foam; the foam including open cell configurations conformable to the wound environment inward of the inner layer; b) a plurality of flexible semiconductors connected to the inner layer, wherein, depending on the direction of a current, one or more of the plurality of flexible semiconductors heats or cools its footprint on the wound environment such that up to about a 25 degree Celsius temperature differential between a first temperature and a second temperature of any of the plurality of semiconductors receiving the current is generated; c) an outer layer adapted to provide uniform distribution of pressure over the wound environment within lumen of tourniquet; the outer layer comprising weaves, wherein on/off application of a second current to the weaves causes outer layer to function as synthetic muscle and deliver micro-increments of compressive, static or decompressive forces to wound environment; d) a communications junction, wherein the communications junction is adapted to releasably hold a communications module; e) the communications module, distinct from the communications junction, comprising a housing adapted for a detachable connection with the communications junction; the housing comprising: i) a first face magnetically reciprocating with the communications junction; ii) a touchscreen; and iii) a combination of a microprocessor, a memory, a visual graphics unit, an audio unit, a transmitter or transceiver adapted for wireless communications; the communications module communicating, via an available wireless cellular network, IEEE 802.11 protocol or military protocol, with a Cloud server or a second computer and a software for controlling the communications module and the tourniquet; and f) circuitry providing connections between the communications module, the communications junction, the sensors, the semiconductors and a power source.

Still another preferred embodiment of the current invention can be described as a detachable communications module adapted for use with a customizable multilayered tourniquet applied to a wound environment; the detachable communications module comprising a housing comprising: a) a first face of the housing adapted to reciprocate magnetically with a communications junction distinct from the housing, wherein the communications junction comprises pins, T-pins, magnets, metallic conductors or electroconductive materials adapted to interface with the first face; b) a combination of a microprocessor, a memory, a visual graphics unit, an audio unit, a transmitter or transceiver, a transducer, a touchscreen, a rechargeable power source and a software for controlling the combination and operations of the customizable multilayered tourniquet, wherein the transmitter or transceiver are adapted for wireless communications, via a wireless cellular network, IEEE 802.11 protocol or military protocol, with a Cloud server or a second computer remote from the communications module; c) circuitry providing connections between the communications module, the communications junction, a plurality of biometric sensors and flexible semiconductors comprising thermal interfaces and ferromagnetic properties proximate the wound environment, an outward layer activatable as synthetic muscle and the rechargeable power source, wherein, depending on the direction of a current, each of the plurality of flexible semiconductors heats or cools its footprint on the wound environment such that up to about a 25 degree Celsius temperature differential between a first temperature and a second temperature of any of the plurality of semiconductors receiving the current is generated; and d) the touchscreen positioned on a second side of the housing visible to a user; the touchscreen displaying interactive data associated with the wound environment to the user; the displayed data allowing the user to control operations of the flexible semiconductors, the biometric sensors and the synthetic muscle of the outward layer.

It is the novel and unique interaction of these simple elements which creates the system within the ambit of the present invention. Pursuant to Title 35 of the United States Code, select preferred embodiments of the current invention follow. However, it is to be understood that the descriptions of the preferred embodiments do not limit the scope of the present invention.

### BRIEF DESCRIPTION OF THE DRAWINGS

FIG. 1 portrays a preferred embodiment of tourniquet (10) with some structures of tourniquet (10) cut away to show an example of a wound environment (200).

FIG. 2 discloses a section of a preferred inner layer of tourniquet (10).

FIG. 3 is an exploded perspective of inner layer (20) and communications module (100).

FIG. 4 portrays communications module (100), Cloud server or first computer (400) and second computer (500) such as a mobile computing device (smart phone, tablet, etc.) or a personal computer or any other type of computing device remote from tourniquet (10).

FIG. 4a portrays touchscreen (140) of communications module (100) that also discloses, in phantom, transducer (108) and LEDs (118) allowing communications module (100) to communicate audibly or visually with the user of the communications module (100).

FIG. 5 is a perspective of outer layer (150) of tourniquet (10) positioned outward of inner layer (20) or bladder (80).

FIG. 6 is another perspective of outer layer (150t) of tourniquet (10).

FIG. 6a is a perspective of rods (158).

FIG. 6b is a perspective of hook and loop fasteners (162)

FIG. 7 is another perspective of outer layer (150) of tourniquet (10).

FIG. 8 is another perspective of outer layer (150) of tourniquet (10).

FIG. 9 is an exploded view of tourniquet (10) that includes bladder (80).

FIG. 10 is perspective of inward side (84) of bladder (80) without first surface (26) and a second surface (28) of inner layer (20).

FIG. 11 portrays rigid semiconductors (24r)

FIG. 11A portrays degrees of flexibility of flexible semiconductors (24f).

FIG. 11B portrays substrate (60s) or substrate (24s) of semiconductors (24) or sensors (60) including carbon nanotubes (68).

FIG. 11C portrays substrate (60s) or substrate (24s) of semiconductors (24) or sensors (60) including carbon graphene lattice (69).

FIG. 12 discloses bag (32) connectable to second port (82) of bladder (80).

FIG. 12A portrays wound environment (200) where fluids (36f, 38f) were applied directly into wound environment (200).

FIG. 13 portrays visual displays of pressures associated with wound environment (200) on touchscreen (140) of communications module (100) or touchscreen (504) of second computer (500).

FIG. 14A portrays visual graphics of temperatures associated with wound environment (200) on touchscreen (140) of communications module (100) or touchscreen (504) of second computer (500).

FIG. 14B portrays visual graphics of temperatures associated with wound environment (200) on touchscreen (140) of communications module (100) or touchscreen (504) of second computer (500).

### DESCRIPTION OF THE PREFERRED EMBODIMENTS

Although the disclosure hereof is detailed to enable those skilled in the art to practice the invention, the embodiments published herein merely exemplify the present invention.

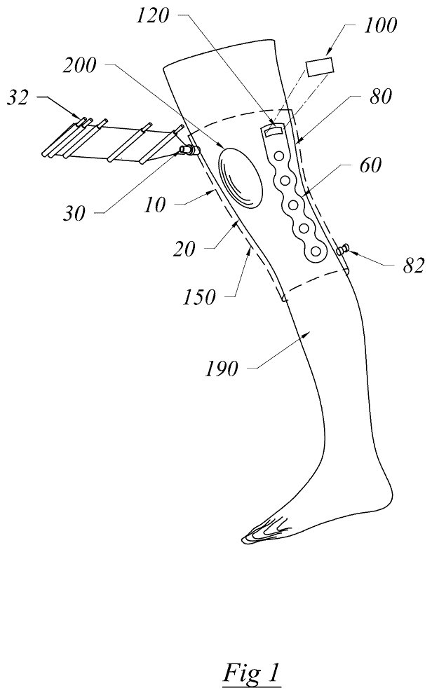

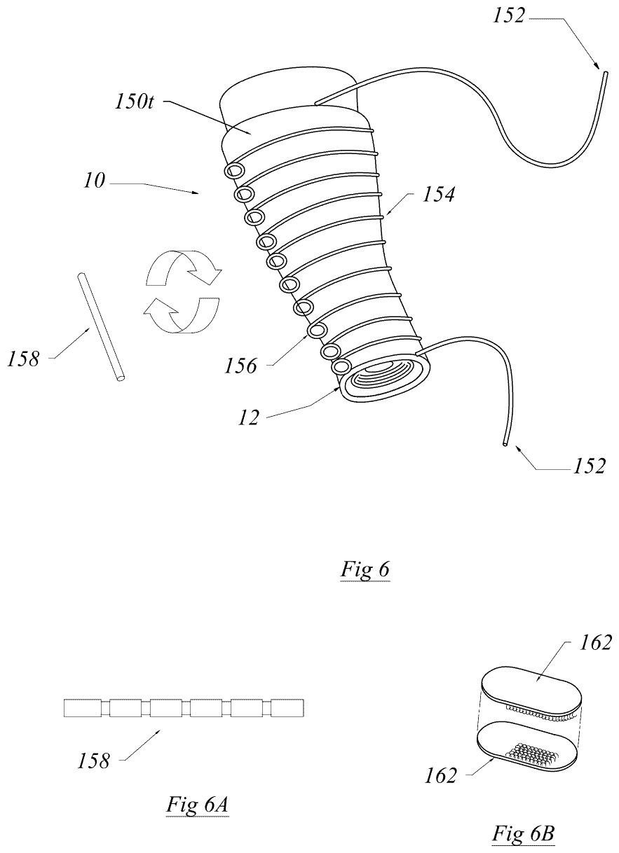

Preferred embodiments of Tourniquet (10) are disclosed in FIGS. 1-8.

FIG. 1 portrays a preferred embodiment of tourniquet (10) applied to patient's extremity (190) with some structures of tourniquet (10) cut away to show an example of a wound environment (200).

The current smart tourniquet (10) includes inner layer (20), outer layer (150) and communications module (100) connected to communications junction (120). Select preferred embodiments can be provided with an optional bladder (80) disposed inward from inner layer (20).

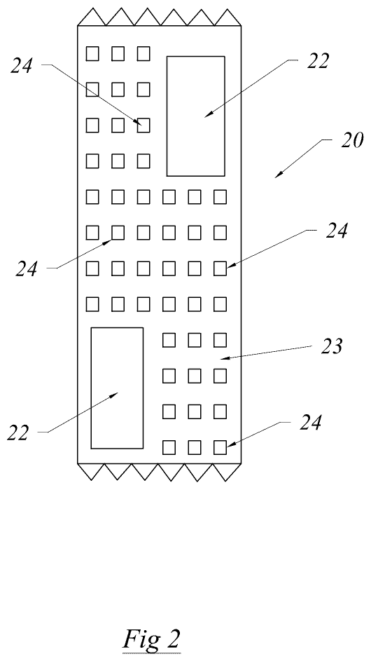

FIG. 2 discloses a section of a preferred inner layer of tourniquet (10). Inner layer (20) can be a flexible, soft and stretchable fabric constructed of one or more polymers or finely knit fabrics. By way of illustration, capillary vessel dilation can occur while absorbing heat away from the skin and inner layer (20) can be provided with a far infrared radiation fabric or bio ceramic-integrated fabric that can improve microcirculation about wound environment (200). Preferred embodiments of inner layer (20) can include hydrophobic adhesive polymers or equivalents (23) that can assist in reducing movements of tourniquet (10) about wound environment (200) of extremity (190). Hydrophobic adhesive polymers or equivalents (23) can be woven into or laminated onto inner layer (20). Inner layer (20) can include compositions impermeable to higher viscosity liquids and fluids. The inner layer (20) can be provided with one or more therapeutic zones (22) including one or more of the following: analgesic, anesthetic, anti-inflammatory, antimicrobial, hemostatic and vasoconstrictive agents, growth factors or any other medically beneficial composition combination thereof.

Select preferred embodiments of inner layer (20) are provided with semiconductors (24), e.g., micro-Peltier units. Tourniquet (10) includes required circuitry (66) interconnecting connecting semiconductors (24) to communications module (100). Current flow direction through semiconductors (24) controls whether semiconductors (24) generate heat or cooling. Footprints of semiconductors (24) can be less than 2 millimeters² to limit potential soft tissues injuries associated with wound environment (200) and nearby skin. Among other things, semiconductors (24) cooling and heating can assist in managing tissue swelling, contraction or secondary defect formation. Direction of a current flowing through the plurality of semiconductors (24) heats or cools designated areas of the wound environment (200). Depending on the direction of current, the plurality of semiconductors (24) can create up to about a 25 degree Celsius temperature differential between a first temperature and a second temperature of any of the plurality of semiconductors (24). The temperature differential can correspond to each semiconductors' (24) footprints associated with the wound environment (200).

When a patient is hypothermic, it may result in microcirculation collapse through higher vascular resistance and lead towards greater soft tissue ischemia or death. Heating the extremity may preserve perfusion to the effected extremity. Alternatively, cooling the extremity may assist in hemostasis and hemorrhage control. Power for semiconductors (24) and other components of the current tourniquet can be provided by batteries, a connection with an alternating current power source or a radio frequency energy supply. Among other things, heat/cool software can generate a heat map of temperatures within tourniquet (10) to better quantify therapeutic intervention required. Whether in the field or the hospital, it is believed that the selective control of temperatures generated by semiconductors (24) inside tourniquet (10) can improve medical outcomes for the patient. Inner layer (20) can be provided with a first port (30) adapted to couple with a device distinct from tourniquet (10) such as bag (32).

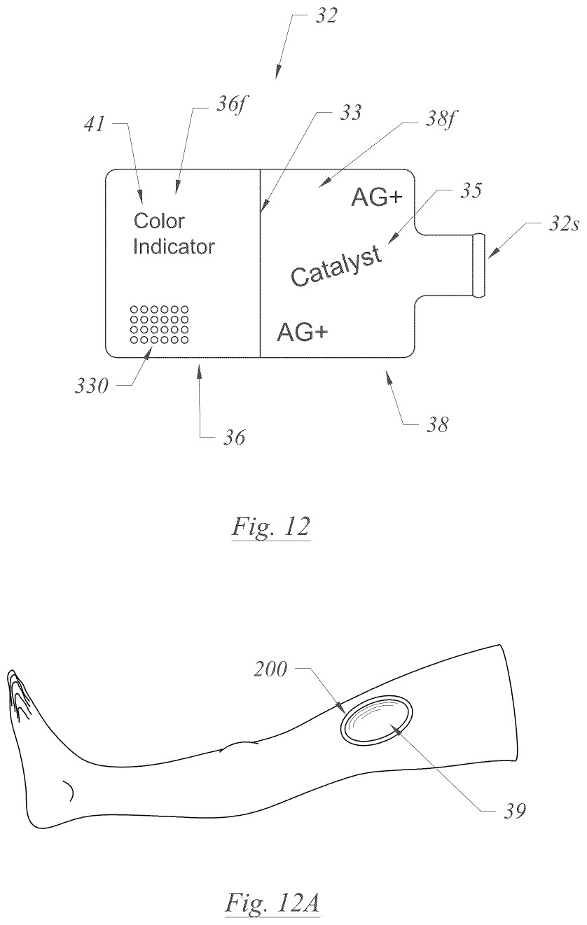

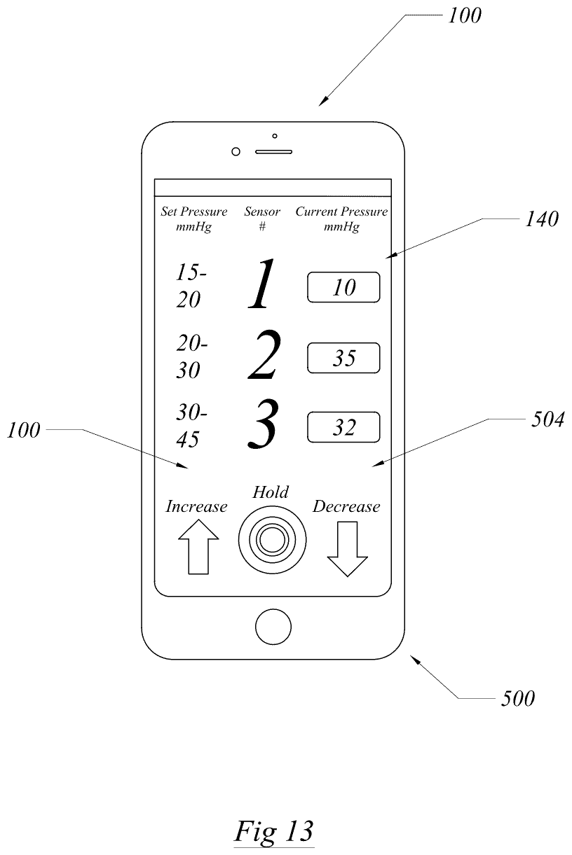

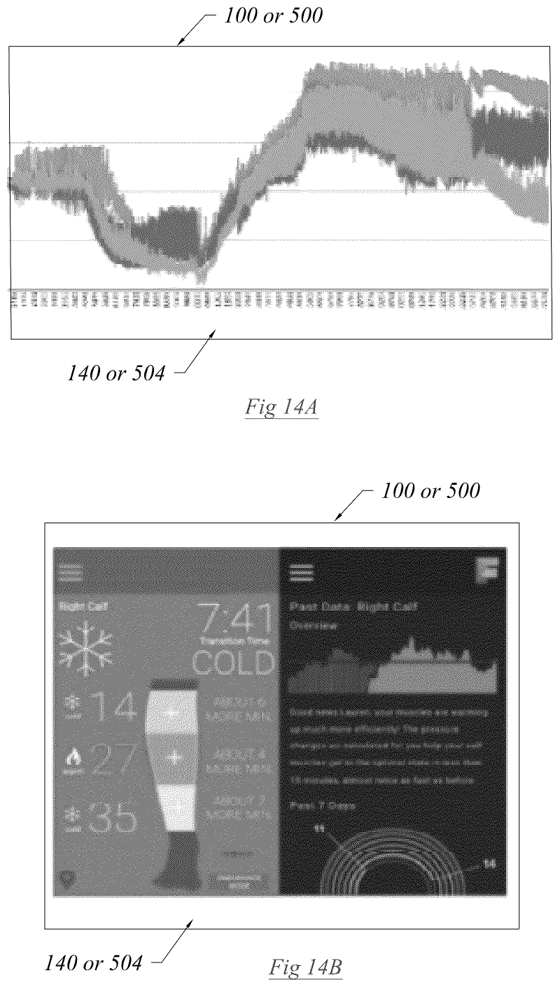

FIG. 12 discloses bag (32) with spout (32s) connectable to optional second port (82) of bladder (80). Via second port (82), bag (32) and spout (32s) can be connected to bladder (80) of tourniquet (10). External bag (32) includes at least two compartmentalized sections (36, 38). Bag (32) is provided with shatterable seal (32s) separating sections (36, 38) and fluids (36f, 38f). When seal (32s) is shattered, fluids (36f, 38f) begin to mix creating a fluid volume that can be injected into wound environment (200) or bladder (80) via second port (82). When the reaction of fluids (36f, 36s) is completed, the fluid volume becomes an expanded or cured foam (39). Among other things, extrusion of different volumes of fluids (36f, 36s) from bag (34) allows custom compression on any morphology of wound environment (200).

Expansion fluids (36f, 38f) can expand about a portion of bladder (90) to create foam (39). Cured foam (39) assists in controlling the pressure applied to the wound environment (200). Bladder (80) can function as an overflow valve for expanded foam (39), that when required can be trimmed and customized subsequent expansion or curing of foam (39). Foaming agents or expansion fluids (36f, 38f) may be composed of silicone, polyurethane, cellulose, bamboo, or other biodegradable agents and will be open cell or an open cell configuration to allow for application of negative pressure or evacuation of fluid or foam (39) when medically required. Foams (39) with open cell configurations allow for therapeutic agents to be applied to the wound environment (200) that can assist with hemostasis and healing. Reactions creating cured foams (39) biocompatible with tissues and are non-exothermic can generate gas. When medical parameters require, an aerosol spray or mixing-tip gun can be utilized to create cured foams (39) for wound environment (200). Bladder (80) provides for selective zonal adjustment of pressures to the wound environment (200).

Use of the current tourniquet (10) allows customized compression on any morphology to wound environment (200), i.e. large defect, amputation, etc. It has been discovered in situ set up of creating a foam layer for an underlying tissue defect can be accomplished in 10 minutes or less.

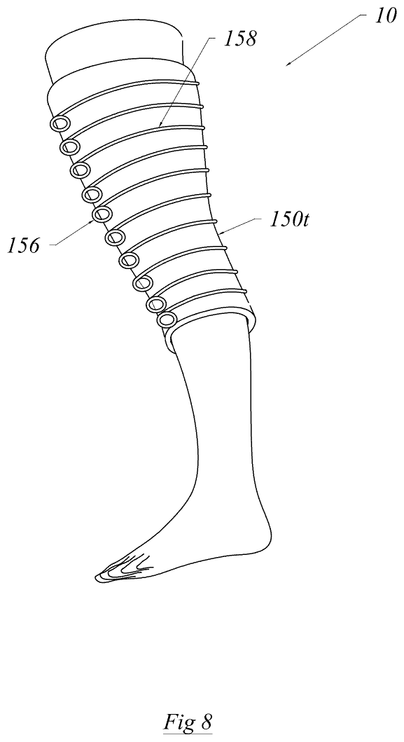

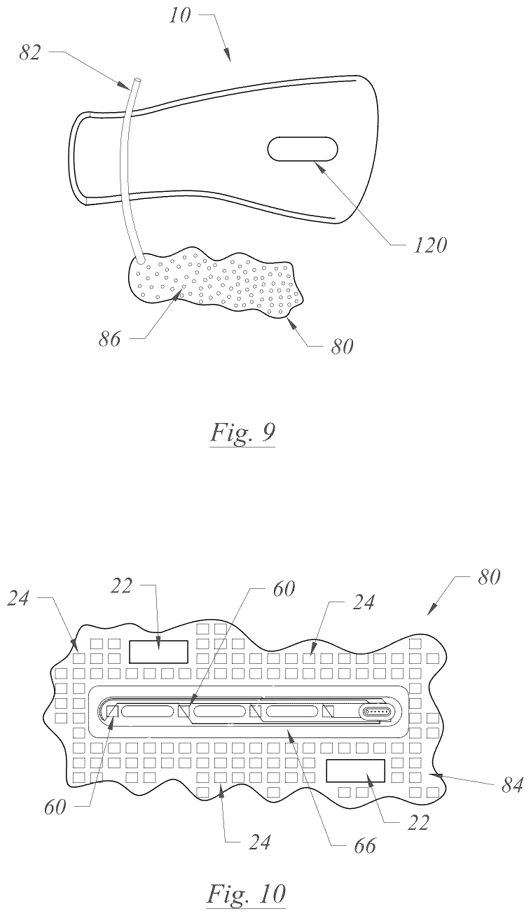

FIGS. 9 and 10 include an exploded view of tourniquet (10) that includes bladder (80) with inward side (84) adapted to face wound environment (200).

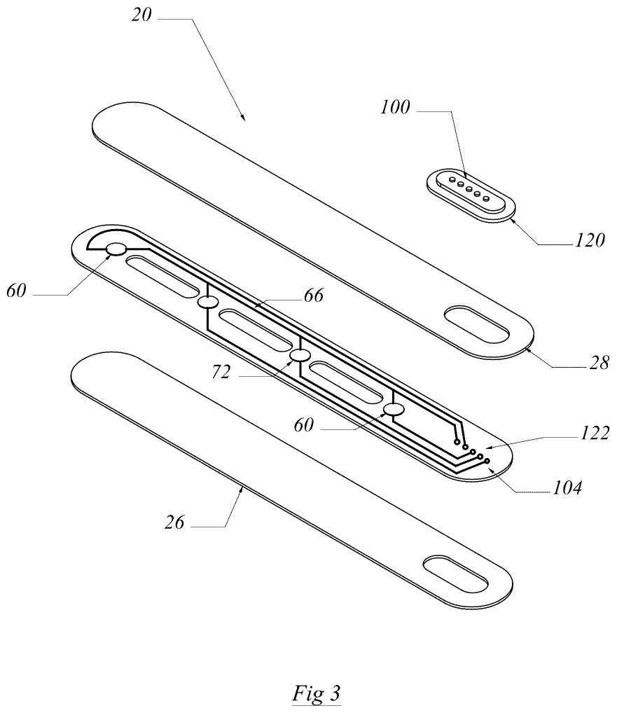

FIG. 3 is an exploded perspective of inner layer (20) and communications module (100). In preferred embodiments of the current invention, sensors (60) are associated with inner layer (20). Sensors (60) can be pressure sensing sensors. When engineering parameters require, sensors (60) can be sandwiched between first surface (26) and a second surface (28) of inner layer (20).

Sensors (60) can be printed onto inner layer (20) or include adhesive layers such as pressure sensitive adhesives (PSA), cement, heat activated polymers. Sensors (60) can be printed directly on inner layer (20) or be transferred on a TPU film through heat activation. For select embodiments, sensors (60) can be composed of electroactive inks (i.e copper, silver, platinum, carbon or combination), graphene lattice (69) or carbon nanotubes (68). One or more sensors (60) are capable of sensing biometrics, including but not limited to, pressure, temperature, blood pressure, lactate levels, pH, sodium levels, potassium level, glucose levels, apoptotic factors, nitric oxide and SVO2. Tourniquet (10) is provided with circuitry (66) connecting the one or more sensors (60) to communications module (100).

FIG. 9 is an exploded view of tourniquet (10) that includes bladder (80). Among other things, this preferred embodiment is provided with communications junction (120), second port (82) and micropores (86).

FIG. 10 is perspective of a section of inward side (84) of bladder (80) without first surface (26) and a second surface (28) of inner layer (20). Inward side (84) can be provided with semiconductors (24), sensors (60), circuitry or leads (66) and therapeutic zones (22).

Bladders (80) can include polymers such as polyurethane (PU) materials. When medical parameters require, bladder (80) or inner layer (24) can include compositions such as graphene lattice (69) or carbon nanotubes (68) capable of acting as synthetic muscle when electric current is supplied to bladder (20) or inner layer (24). Use of synthetic muscle can provide increased compression, electronically controlled compression, or sequential compression of inner layer (20). Although not shown in the Drawings, tourniquet (10) is provided with circuitry adapted to connect synthetic muscle to communications module (100).

Tourniquet (10) is provided with communications junction (120) adapted to releasably hold communications module (100). Communications junction (120) can be distinct from inner layer (20) and composed of TPU materials, silicone, PU or other materials acceptable in the art. Communications junction (120) is provided with a junction for electrical connections and interconnected with some or all of the circuitry of tourniquet (10). In select preferred embodiments, communications junction (120) can be provided with one or more lips or rails to releasably hold communications module (100) to tourniquet (10). Among other things, removability of communications module (100) from tourniquet (10) allows cleaning or changing one or more layers (20, 80) without damaging communications module (100).

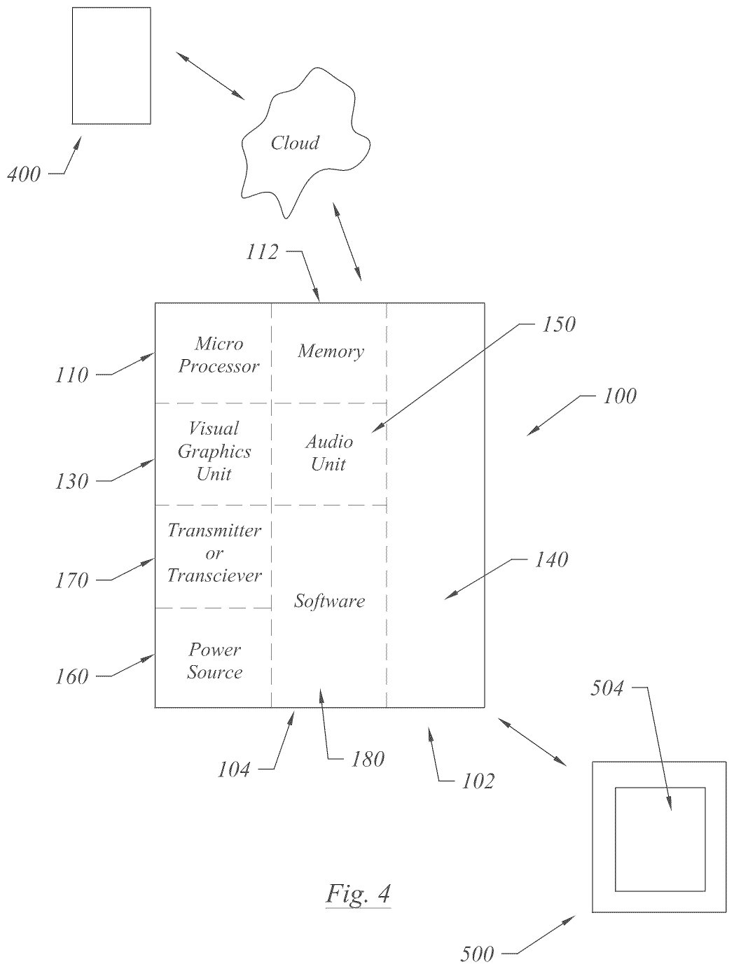

FIG. 4 portrays communications module (100), Cloud server or computer (400) and second computer (500) such as a mobile computing device (smart phone, tablet, etc.) or a personal computer or any other type of computing device remote from tourniquet (10).

FIG. 4a portrays touchscreen (140) of communications module (100) that also discloses, in phantom, transducer (108) and LEDs (118) allowing communications module (100) to communicate audibly or visually with the user of the communications module (100). Visual graphic unit (130) controls visual communications between the communications module (100) and the user. Audio unit (150) controls audible communications between the communications module (100) and the user.

Within the ambit of the current invention, communications module (100) can be provided with one or more of the following components: microprocessor (110), memory (112), visual graphics unit (130), touchscreen (140), audio unit (150), power source (160), transmitter or transceiver (170), circuitry interconnecting the components and software (180) for controlling the components. Housing (102) of communications module (100) can include interface (104) adapted to communicate with circuity (66) and junction of inner layer (20). Examples of potential interfaces (104) include pins, T-pins, magnets (122), metallic conductors or electroconductive materials. Touchscreen (140) can be an OLED or AMOLED touch screen (140) incorporated into an outward face of communications module (100). Within the scope of the current invention, tourniquet (10) can generate a visual or audible alarm when calculated preselected medical parameters are outside predetermined ranges.

Power sources (160) for communications module (100) include but are not limited to rechargeable sources such as lithium ion, lithium iron phosphate, solid state, tab-less batteries or other usable power sources. Within the scope of the invention, the power source can be attached to housing (102). The rechargeable power sources can be detachable from housing (102) to engage the recharging energy supply.

Communications module (100) of tourniquet (10) is adapted for wireless communications via any wireless network such as available cellular networks and/or IEEE 802.11 protocol at a frequency of 2.4 GHz (Wi-Fi or Bluetooth) or military protocol. Transmitter or transceiver (170) can communicate with a Cloud server or computer (400), second computer (500) such as a mobile computing device (smart phone, tablet, etc.) or a personal computer or any type of computing device remote from tourniquet (10). The combination of software (180), memory (112) and transmitter/transceiver (170) allow communications to and from Cloud server (400) and/or second computer (500) to control or assist in controlling medical parameters of tourniquet (10) relative to the wound environment (200). Communications from the Cloud server (400) or second computer (500) can be visualized on touchscreen (140) or heard via transducer (180). Devices remote from tourniquet (10) can be utilized to store data, calculate biometrics and/or control pressures applied by tourniquet (10) to the wound environment (200). By way of illustration, calculations of sensed biometric data can generate an alarm alerting the user of prolonged ischemic times associated with wound environment (200). Tourniquet (10) can also be programmed to perform its functions separately from Cloud server (400) and/or second computer (400).

In other preferred embodiments of tourniquet (10), communications module (100) can also calculate biometrics and/or control pressures applied by tourniquet (10) to the wound environment (200). Within in the scope of the current invention, sensors (60) can be provided with transmitters or transceivers (72) for direct wireless communications with communications module (100) or other computing device remote from tourniquet (10).

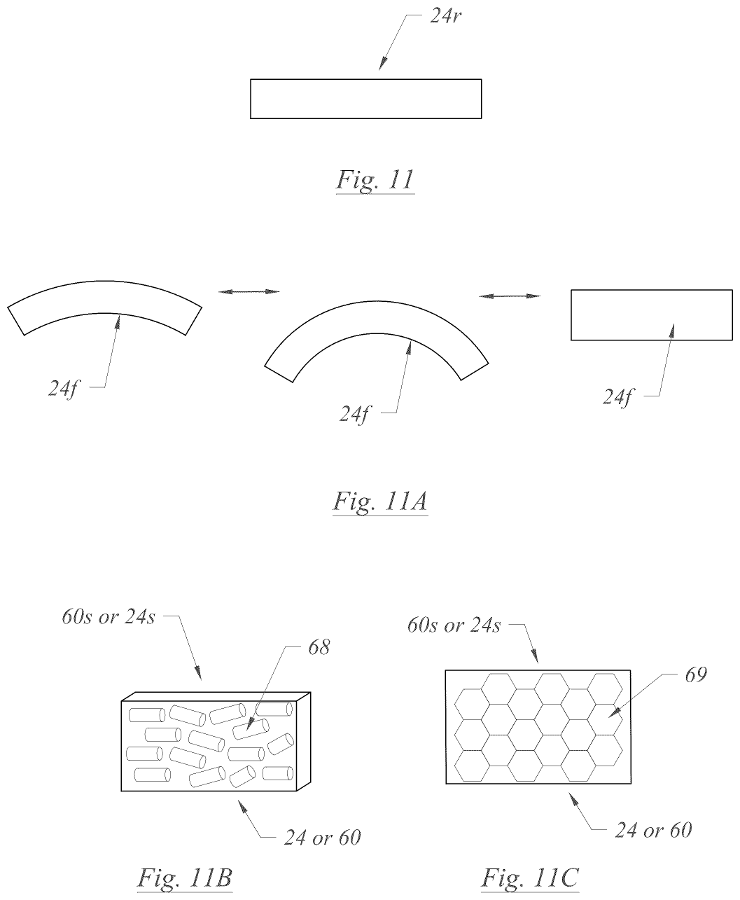

FIG. 11 portrays rigid semiconductors (24r) and flexible semiconductors (24s). Depending on medical parameters of the patient, tourniquet (10) can utilize flexible semiconductors (24f), rigid conductors (24r) or a combination thereof.

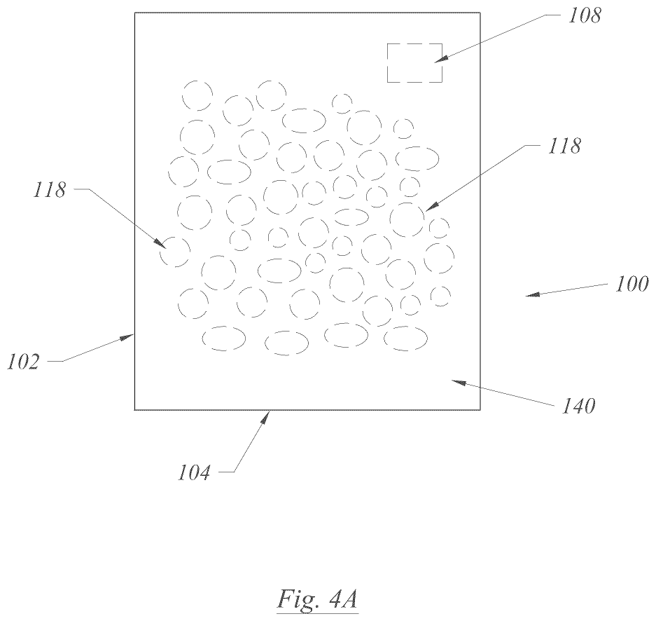

FIG. 5 is a perspective of outer layer (150t) of tourniquet (10) positioned outward of inner layer (20) or bladder (80). Outer layer (150t) can be composed of compressive materials and or woven fabrics capable of providing uniform distribution of pressure over soft tissue within lumen (12) of tourniquet (10). Cords (152) are incorporated with outer layer (150t) and utilized to manually control compressive forces supplied by inner layer (20). In select preferred embodiments, outer layer (150t) can include a dial apparatus (BOA® system) for cords (152) that can provide micro-control of compressive forces supplied to inner layer (20). The compressive materials can include far infrared technologies adapted to assist in improving microcirculation about wound environment (200). In select preferred embodiments of current tourniquet (10), on/off application of electric current to weaves of outer layer (150t) can cause outer layer (150t) to function as synthetic muscle and deliver micro-increments of compressive, static or decompressive forces to wound environment (200) or injured tissue. It is believed that outer layer's (150t) provision of gentle compression assists with venous hemostasis and lessons venous hypertension. When medical parameters require, semiconductors (24) or other communications hardware can be incorporated into outer layer (150t). Outer layer (150t) can include components for communication with communications module (100).

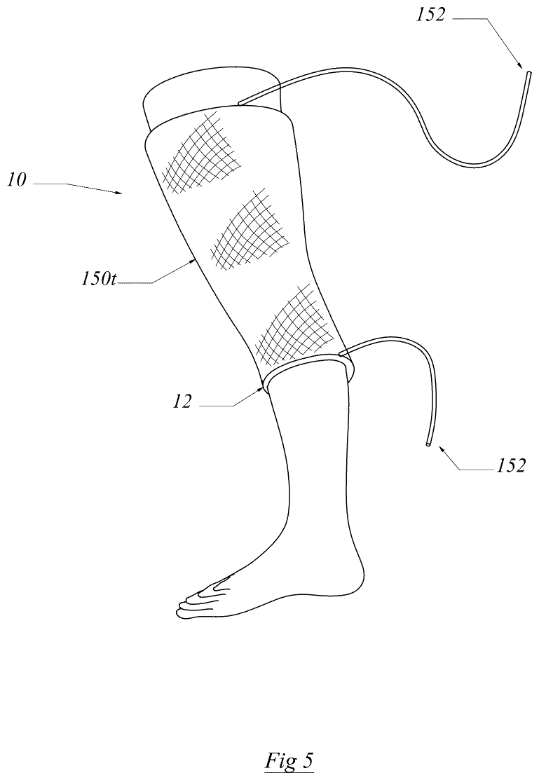

FIG. 6 is another perspective of outer layer (150t) of tourniquet (10) applied to patient (P). Outer layer (150t) can be provided with rings (154) with loops (156). This embodiment is intended for use with larger vessel diameter compression and intervention such as arteries and larger bore or diameter veins. In operation, one or more rods (158) are inserted through loops (156). Twisting or rotation of rods (158) can be performed (distal to proximal) to one or more loops (156) until the bleeding is stopped. Twisted rods (158) can be secured by hook and loop fasteners (162) attachable to outer layer (150). Rods (158) can be composed of lightweight materials (carbon fiber, nylons, nylon with glass, quartz tpt, etc). Rods (158) can be segmented and connected, multiple and/or collapsible for ease of storage. When rods (158) are not twisted, one or more rods (158) can provide segmental and rigid or semi-rigid fixation to the coronal plane to the patient's extremity (190). Rods (158) can be locked for providing rigid support along outer layer (150) of tourniquet (10). Segments of rods (158) can function as a splint. Select preferred embodiments of outer layer (150) can include a pocket for storage. Rings (154) can be composed of semi-rigid polymers or fabrics. One or more loops (156) can be provided with pressure sensors.

User interactive visual graphics of the calculated and correlated pressures, temperatures and sensed biometrics are visible on touch screen (140) of communications module (100) and/or computing devices (400, 500) remote from tourniquet (10). In select embodiments, the visual graphics can be color coded. Within the scope of the current invention, touchscreen (140) can display interactive data correlated and sensed by sensors (50) and semiconductors (60). The interactive, visualized, calculated and correlated data can be supplied to detachable communications module (100), cloud server (400) or computer remote (500) remote from communications module (100) or a combination thereof allowing the user of touchscreen (140) to control application of pressure to wound environment (200). Pressures supplied to wound environment (200) can also be controlled by cloud server (400) or computer remote (500) remote from communications module (100). Touchscreen (140) can also display interactive correlated data from biometric sensors (60) sensing one or more of pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2. Touchscreen (140) can also display a pressure map or a heat map or both associated with wound environment (200) or injured tissue.

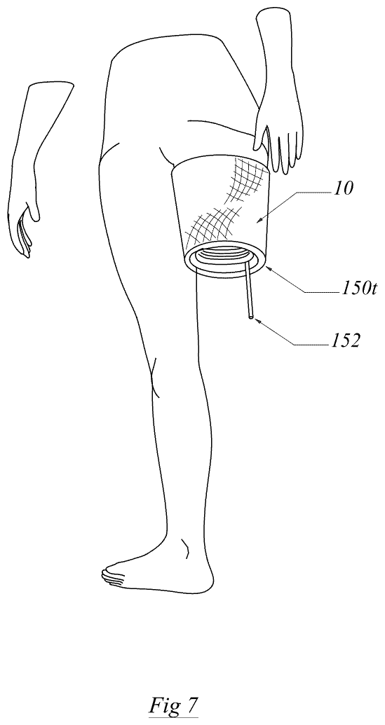

With reference to FIG. 8, another preferred embodiment of tourniquet (10) shows that outer layer (150t) is provided with segmented rods (158) and loops (156). Segmented rods (158) can be utilized as one or more splints on one or more sides of outer layer (150t). It is believed that his embodiment can be useful to support an extremity (190) of patient (P) having a femur fracture. Although not shown in the Drawings, tourniquet (10) can include a zipper spanning the length of tourniquet (10) for quicker application to an extremity. Other embodiments of tourniquet (10) can be provided with straps, hook and loop fasteners, hooks and catches, zippers or any combination to secure the tourniquet about the wound environment and improve the tourniquet's post application stability.

When medical parameters require, tourniquet (10) can be folded back on itself to generate additional pressure or compression. When required, one or more longitudinal ends of tourniquet (10) can be closed off for use in a stump compression. For a below the knee amputation, length of tourniquet (10) is adequate to extend beyond the knee. Tourniquet (10) is portable, flat, lightweight, transportable and is adapted for use in in hospital and field settings.

Select preferred embodiments of the current invention have been disclosed and enabled as required by Title 35 of the United States Code.

What is claimed is:

1. A tourniquet adapted for use about a wound environment or an injured tissue; the tourniquet comprising:
a) a bladder, comprising:
i) an inward side fitted to contact a contour of a portion the wound environment and adapted to receive an expansion fluid for supplying a preselected pressure to the wound environment, wherein:
ii) the expansion fluid includes polymerized beads;
iii) a first current flowing through graphene or carbon nanotubes causes the bladder to function as a first synthetic muscle for the wound environment; and
iv) a port;
b) an inner layer comprising an infrared radiation fabric or a bio ceramic-integrated fabric; the inner layer adapted to be proximate the bladder and the wound environment; the inner layer further comprising a hydrophobic adhesive polymer for impeding movement of the tourniquet about the wound environment;
c) an array comprising:
i) a group of first sensors conformable to the contour of a portion the wound environment and a section of the bladder, wherein the group of first sensors is adapted to sense pressures associated with the wound environment; and
ii) a communications junction comprising a first magnetic source;
d) a plurality of semiconductors comprising thermal interfaces and ferromagnetic properties connected to a wound environment facing side of the bladder, wherein dependent on a direction of a second current flowing through the thermal interfaces, the second current causes at least some of the plurality of semiconductors to heat or cool an area of the wound environment associated with the at least some of the plurality of semiconductors' footprints;
e) an outer layer surrounding an outward side of the inner layer;
f) a detachable communications module adapted for connection to the communications junction comprising a housing comprising:
i) a first interface magnetically reciprocating with the communications junction;
ii) a touchscreen visible to a user; and
iii) a combination of components comprising a microprocessor, a memory, a visual graphics unit, an audio unit, a transmitter or transceiver and a software controlling the detachable communications module;
g) the software:
i) communicating with the group of first sensors and the plurality of semiconductors; and
ii) controlling the microprocessor, the memory, the visual graphics unit, the audio unit, the transmitter or the transceiver and visual displays projected by the touchscreen, wherein the software controlling the detachable communications module is located on the detachable communications module, a Cloud server or a second computer remote from the detachable communications module, or a combination thereof;
h) the transmitter or transceiver adapted for wireless communications, via an available wireless cellular network, an IEEE 802.11 protocol or military protocol with a Cloud server or a second computer remote from the communications module; and
i) circuitry providing connections between the detachable communications module, the communications junction, the group of first sensors and the plurality of semiconductors and a power source for the detachable communications module.

2. The tourniquet of claim 1, wherein at least some of the plurality of sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors or SVO2.

3. The tourniquet of claim 2 further comprising an alarm, wherein the alarm is audible, visual or a combination thereof when the sensed biometric for a patient is outside of a predetermined range.

4. The tourniquet of claim 2, wherein the touchscreen portrays a pressure map or a heat map or both associated with the wound environment.

5. The tourniquet of claim 4, wherein the inner layer comprises one or more therapeutic zones including one or more of the following compositions: analgesic, anesthetic, anti-inflammatory, antimicrobial, hemostatic and vasoconstrictive agents, growth factors or a combination thereof.

6. The tourniquet of claim 5, wherein up to about a 25 degree Celsius temperature differential between a first temperature and a second temperature of any of the plurality of semiconductors receiving the second current.

7. The tourniquet of claim 1, wherein the semiconductors are rigid or flexible or a combination thereof.

8. A tourniquet adapted for use about a wound environment; the tourniquet comprising:
a) an inner layer comprising:
i) an infrared radiation fabric or a bio ceramic-integrated fabric;
ii) a plurality of sensors attached to the inner layer, wherein at least some of the plurality of sensors sense pressures associated with the wound environment; and
ii) a port adapted to receive and distribute expansion fluids, including polymerized beads, creating a foam; the foam including open cell configurations conformable to the wound environment inward of the inner layer;
b) a plurality of flexible semiconductors connected to the inner layer, wherein, depending on the direction of a current, one or more of the plurality of flexible semiconductors heats or cools its footprint on the wound environment such that up to about a 25 degree Celsius temperature differential between a first temperature and a second temperature of any of the plurality of semiconductors receiving the current is generated;
c) an outer layer adapted to provide uniform distribution of pressure over the wound environment within lumen of tourniquet; the outer layer comprising weaves, wherein on/off application of a second current to the weaves causes outer layer to function as synthetic muscle and deliver micro-increments of compressive, static or decompressive forces to wound environment;
d) a communications junction, wherein the communications junction is adapted to releasably hold a communications module;
e) the communications module, distinct from the communications junction, comprising a housing adapted for a detachable connection with the communications junction; the housing comprising:
i) a first face magnetically reciprocating with the communications junction;
ii) a touchscreen; and
iii) a combination of a microprocessor, a memory, a visual graphics unit, an audio unit, a transmitter or transceiver adapted for wireless communications; the communications module communicating, via an available wireless cellular network, IEEE 802.11 protocol or military protocol, with a Cloud server or a second computer and a software for controlling the communications module and the tourniquet; and
f) circuitry providing connections between the communications module, the communications junction, the sensors, the semiconductors and a power source.

9. The tourniquet of claim 8, wherein at least some of the plurality of sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2.

10. The tourniquet of claim 9, wherein the inner layer comprises one or more therapeutic zones including one or more of the following compositions: analgesic, anesthetic, anti-inflammatory, antimicrobial, hemostatic and vasoconstrictive agents, growth factors or a combination thereof.

11. The tourniquet of claim 10 further comprising an alarm, wherein the alarm is visualized on touchscreen or heard via transducer.

12. The tourniquet of 11, wherein the display portrays a pressure map or a heat map or both associated with the wound environment.

13. The tourniquet of claim 12 further comprising one or more straps, hook and loop fasteners, hooks and catches, zippers or any combination thereof.

14. A detachable communications module adapted for use with a customizable multilayered tourniquet applied to a wound environment; the detachable communications module comprising a housing comprising:
a) a first face of the housing adapted to reciprocate magnetically with a communications junction distinct from the housing, wherein the communications junction comprises pins, T-pins, magnets, metallic conductors or electroconductive materials adapted to interface with the first face;
b) a combination of a microprocessor, a memory, a visual graphics unit, an audio unit, a transmitter or transceiver, a transducer, a touchscreen, a rechargeable power source and a software for controlling the combination and operations of the customizable multilayered tourniquet, wherein the transmitter or transceiver are adapted for wireless communications, via a wireless cellular network, IEEE 802.11 protocol or military protocol, with a Cloud server or a second computer remote from the communications module;
c) circuitry providing connections between the communications module, the communications junction, a plurality of biometric sensors and flexible semiconductors comprising thermal interfaces and ferromagnetic properties proximate the wound environment, an outward layer activatable as synthetic muscle and the rechargeable power source, wherein, depending on the direction of a current, each of the plurality of flexible semiconductors heats or cools its footprint on the wound environment such that up to about a 25 degree Celsius temperature differential between a first temperature and a second temperature of any of the plurality of semiconductors receiving the current is generated; and
d) the touchscreen positioned on a second side of the housing visible to a user; the touchscreen displaying interactive data associated with the wound environment to the user; the displayed data allowing the user to control operations of the flexible semiconductors, the biometric sensors and the synthetic muscle of the outward layer.

15. The detachable communications module of claim 14, wherein the displayed data on the touchscreen allows the user of the communications module to control:
a) delivery of micro-increments of compressive, static or decompressive forces to the wound environment; and/or
b) heating or cooling footprints supplied by one or more flexible semiconductors to the wound environment.

16. The detachable communications module of claim 14, wherein the biometric sensors sense one or more of the following biometrics: pressure, temperature, blood pressure, lactate levels, pH, growth factors, hydrogen levels, sodium levels, potassium level, lactate levels, other electrolytes, cytokines, glucose levels, apoptotic factors, nitric oxide or SVO2.

17. The detachable communications module of claim 16, wherein the displayed data was created by the communications module, a Cloud server, the second computer remote from the communications module or a combination thereof.

18. The detachable communications module of claim 16 further comprising an audible, visual or a combination thereof alarm activated when the sensed biometric is outside of a predetermined range.
